(19) United States
(12) Patent Application Publication
(10) Pub. No.: US 2004/0053311 A1
(43) Pub. Date: Mar. 18, 2004

(54) Methods for identifying phenotype and protein activity inhibitors

(76) Inventors: Rami Skaliter, Nes Ziona (IL); Paz Einat, Nes Ziona (IL)

Correspondence Address: Cooper & Dunham LLP, 1185 Avenue of the Americas, New York, NY, 10036, US

(21) Appl. No.: 10/620,193

(22) Filed: Jul. 15, 2003

Related U.S. Application Data
(60) Provisional application No. 60/396,279, filed on Jul. 15, 2002.
(60) Provisional application No. 60/449,757, filed on Feb. 25, 2003.

(51) Int. Cl. C12Q 1/68; G01N 33/53; C12M 1/34

(52) U.S. Cl. 435/6; 435/7.1; 435/287.2

(57) ABSTRACT

The present invention discloses screening processes aimed at identifying phenotype and protein activity inhibitors. In addition, processes for identifying small molecule protein inhibitors are provided. In some aspects, the invention employs microarray technology.

This application claims the benefit of U.S. provisional patent application No. 60/396279, filed 15, Jul. 2002, and of U.S. provisional patent application No. 60/449757, filed 25, Feb. 2003, which are both hereby incorporated by reference in their entirety.

### FIELD OF THE INVENTION

The present invention relates to molecular biology screening processes and to protein screening and targeting.

### BACKGROUND OF THE INVENTION

The identification and validation of “druggable” protein targets for drug screening is a difficult and complicated process; protein domains which may be susceptible to inhibitory targeting and/or druggable are not always easy to identify, and thus, drug design can be problematic. The present invention provides a novel method for identifying protein inhibitors, by way of targeting protein domains suitable for drug screening. More specifically, the present invention employs active polypeptide fragments in screening systems, preferably inhibitor screening systems.

Active Polypeptide Fragments (APFs)

An active polypeptide fragment is derived from a naturally occurring or synthetically modified polypeptide or a portion of a polypeptide, and possesses activity which agonizes or antagonizes the activity of a naturally occurring polypeptide. Specifically, an active polypeptide fragment may modulate the activity of the naturally occurring polypeptide from which it is derived, a polypeptide of the same family or a homologous polypeptide, or even a non-homologous polypeptide related to the active polypeptide fragment by direct (e.g., an antibody or a receptor) or indirect (through an adaptor molecule) binding. The modulation may occur at the level of the polypeptide, by directly acting on the polypeptide or via interaction with a molecule with which said polypeptide interacts; the modulation may also occur at the level of the polynucleotide, by modulating the transcription or translation of the polynucleotide (via direct or indirect interaction). Examples of active polypeptide fragments include, but are not limited to, antibodies or fragments of antibodies (see below), polypeptide fragments comprising an active site or a portion thereof of a polypeptide, dominant negative peptides, dominant positive peptides, and any of these polypeptide fragments which have been synthetically or chemically modified.

The preparation and use of APFs is detailed in Example 1.

Microarray Technology

An array is an orderly arrangement of samples. It provides a medium for matching known and unknown samples based on base-pairing or biochemical interactions and automating the process of identifying the unknowns. An array experiment can make use of common assay systems such as microplates or standard blotting membranes, and can be created by hand or by use of robotics to deposit the sample. In general, arrays are described as macroarrays or microarrays, the difference being the size of the sample spots. Microarrays require specialized robotics and imaging equipment.

DNA microarrays, also termed DNA chips, are fabricated by high-speed robotics, generally on glass but sometimes on nylon substrates, for which probes with known identity are used to determine complementary binding, thus allowing massively parallel gene expression and gene discovery studies. An experiment with a single DNA chip can provide researchers information on thousands of genes simultaneously—a dramatic increase in throughput compared to earlier methods.

There are two major applications for DNA microarray technology: 1) Identification of sequence (gene/gene mutation); and 2) Determination of expression level (abundance) of genes. An additional application for microarray technology is that of the Achilles Heel Method—see U.S. Pat. No. 6,057,111, co-assigned to applicants.

There are several steps in the design and implementation of a DNA microarray experiment, and many innovations have been developed regarding each of the following steps: 1) DNA types; 2) Chip fabrication; 3) Sample preparation; 4) Assay; 5) Readout; and 6) Software (informatics)

Applications of DNA microarray technology include: Gene discovery; disease diagnosis; drug discovery (Pharmacogenomics) and toxicological research (Toxicogenomics).

Production and use of DNA microarrays is well-known in the art and is described inter alia in U.S. Pat. Nos. 6,291,170; 5,807,522 (inventors Shalon and Brown); and in U.S. Pat. Nos. 6,110,426; 5,716,785 and 5,891,636, (inventors Eberwine et al) all assigned to Board of Trustees of Leland Stanford University and in U.S. Pat. No. 4,981,783 (inventor Augenlicht).

Protein microarrays are also known in the art. See,: for example, Ekins R. P., J Pharm Biomed Anal 1989. 7: 155; Ekins R. P. and Chu F. W., Clin Chem 1991. 37: 1955; Ekins R. P. and Chu F. W, Trends in Biotechnology, 1999, 17, 217-218)

The are two main objectives for proteomic research: 1. quantification of the proteins expressed in a cell; 2. functional study of thousands of proteins in parallel. For quantification purposes, the standard method is 2D gel separation followed by Mass-Spectrometry identification. For protein function study, microarray-based assays are being used to study protein-protein and protein-ligand interactions.

A recent scientific paper (G. MacBeath and S. L. Schreiber, Printing Proteins as Microarrays for High-Throughput Function Determination, Science 2000 Sep. 8; 289(5485): p. 1760-1763) describes a protein microarray on which more than 10,000 protein spots were printed on a glass slide. The chip was used to identify protein-protein and protein-drug interactions. This could represent a major breakthrough in proteomics and drug discovery, but there still remains the problem of obtaining thousands of pure proteins and maintaining them in their natural conformation.

Chemical Microarrays

In addition, recent studies suggest that it is possible to conduct experiments using microarrays imprinted with chemicals/small molecules.

Graffinity Pharmaceutical Design GmbH of Heidelberg, Germany uses chemical microarrays as screening tools to enhance the understanding of protein binding specificity, based on diversity Label-free Detection. The drug discovery process is based on a chemical microarray approach, in which small molecules are immobilized on a carrier surface and provided in a screening-ready standardized format. Incubation of the immobilized compounds with the purified and solubilized target protein yields comprehensive affinity fingerprints in a label- and assay-free procedure. Graffinity apparently has proprietary low molecular weight compound libraries for chemical microarrays which are based on small drug fragments; the chemical microarrays are apparently also useful for the examination of protein families.

Additional publications on the preparation and screening of small molecule microarrays are: Gregory A. Korbel, Gojko Lalic, and Matthew D. Shair: Reaction Microarrays: A Method for Rapidly Determining the Enantiomeric Excess of Thousands of Samples, Journal of the American Chemical Society; 2001; 123(2); 361-362; Paul J. Hergenrother, Kristopher M. Depew, and Stuart L. Schreiber: Small-Molecule Microarrays: Covalent Attachment and Screening of Alcohol-Containing Small Molecules on Glass Slides, Journal of the American Chemical Society; 2000; 122(32); 7849-7850; Gavin MacBeath, Angela N. Koehler, and Stuart L. Schreiber: Printing Small Molecules as Microarrays and Detecting Protein-Ligand Interactions en Masse, Journal of the American Chemical Society; 1999; 121(34); 7967-7968.

Small molecule microarrays have also been used to identify small molecules that bind to a protein of interest e.g. as described by Kuruvilla et al, Nature 416 (2002), 653-657.

PCT patent application publication numbers WO 99/13313 and WO 02/05945 disclose different methods for producing microarray chips imprinted with a variety of test substrates; in addition, WO 00/71746 discloses a method of producing a microarray of chemical compounds.

Although APFs can mimic the active site of the parent protein, no publication thus far has suggested linking APFs to a screening system as the screening agent. The inventors of the present invention propose to utilize APFs as a probe in known screening systems; this should enable isolation of compounds that specifically inhibit the parent protein and target domains associated with its biological activity, and thus may offer advances in drug design.

### SUMMARY OF THE INVENTION

The present invention discloses novel screening methods for identifying protein activity inhibitors and phenotype inhibitors. More specifically, the present invention relates to the use of active polypeptide fragments in screening systems.

In one embodiment, the present invention provides a process for obtaining a species which modulates the activity of a polypeptide.

Another embodiment of the present invention concerns a process of obtaining a species that modulates a phenotype.

An additional embodiment of the present invention provides a process of obtaining a small-molecule polypeptide inhibitor.

### DETAILED DESCRIPTION OF THE INVENTION

One embodiment of the present invention provides a process of obtaining a species which inhibits the activity of a polypeptide, comprising:

a) contacting an active polypeptide fragment of the polypeptide with the species;

b) determining if the species interacts with the active polypeptide fragment; and

c) determining if the species inhibits the activity of the polypeptide

This process may in addition be employed in order to obtain enhancers of the activity of a polypeptide. Further, the process may be performed with a plurality of species and/or a plurality of active polypeptide fragments.

The species may be a chemical compound, a polypeptide, a polynucleotide, or any other molecule potentially possessing modulating abilities. The active polypeptide fragment may be a polypeptide fragment comprising the active site or a portion thereof of a polypeptide of interest, or a dominant polypeptide fragment.

In an exemplary embodiment, the species are chemical compounds imprinted on an array and the active polypeptide fragment is a dominant polypeptide fragment or a polypeptide fragment comprising an active site of one or more polypeptide of interest.

In general, the term “species” encompasses, inter alia, small chemical molecules, antibodies, antisense oligonucleotides, antisense DNA or RNA molecules, siRNAs, proteins, polypeptides and peptides including peptido-mimetics, expression vectors, lipids, carbohydrates and any other molecule capable of interacting with a naturally occurring molecule or synthetic or semi-synthetic natural products. In addition, the species may be imprinted on a microarray.

The term “imprinted on a microarray” refers to molecules which are printed and/or immobilized on an array; this can be accomplished according to methods known in the art, essentially as disclosed herein.

By the term “antisense” (AS) or “antisense fragment” is meant a nucleic acid fragment having inhibitory antisense activity, said activity causing a decrease in the expression of the endogenous gene. The sequence of the AS is designed to complement a target mRNA of interest and form an RNA: AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, 1996: Antisense strategies in the treatment of leukemias. Semin Oncol. 23(1):78-87). In that case, RNase H will cleave the RNA component of the duplex and can potentially release the AS to further hybridize with additional molecules of the target RNA. An additional mode of action results from the interaction of AS with genomic DNA to form a triple helix which can be transcriptionally inactive.

The terms “chemical compound”, “small molecule”, “chemical molecule” “small chemical molecule” and “small chemical compound” are used interchangeably herein and are understood to refer to chemical moieties of any particular type which may be synthetically produced or obtained from natural sources and typically have a molecular weight of less than 2000 daltons, more preferably less than 1000 daltons or even less than 600 daltons.

By the term “polypeptide” is meant a molecule composed of amino acids and the term includes peptides, polypeptides, proteins and peptidomimetics; active polypeptide fragments are also encompassed by this term.

A “peptidomimetic” is a compound containing non-peptidic structural elements that is capable of mimicking the biological action(s) of a natural parent peptide. Some of the classical peptide characteristics such as enzymatically scissille peptidic bonds are normally not present in a peptidomimetic.

The term “amino acid” refers to a molecule which consists of any one of the 20 naturally occurring amino acids, amino acids which have been chemically modified, or synthetic amino acids.

“Chemically modified”—when referring to the product of the invention, means a product (polypeptide) where at least one of its amino acid residues is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

The term “polynucleotide” refers to any molecule which comprises two or more of the bases guanidine, citosine, timidine, adenine, uracil or inosine, inter alia, or chemical analogs thereof, includes “oligonucleotides” and encompasses “nucleic acids”.

By “silencing RNA” (siRNA) is meant an RNA molecule which decreases or silences the expression of a gene/mRNA of its endogenous or cellular counterpart. The term is understood to encompass “RNA interference” (RNAi), and “double-stranded RNA” (dsRNA). For recent information on these terms and proposed mechanisms, see Bernstein E., Denli A M., Hannon G J: The rest is silence. RNA. 2001 ; November;7(11):1509-21; and Nishikura K.: A short primer on RNAi: RNA-directed RNA polymerase acts as a key catalyst. Cell. 2001 Nov. 16; 107(4):415-8.

A “modulator” is any molecule that is capable of modulation, i.e. that either increases (promotes) or decreases (prevents). The term is understood to include partial or full inhibition, stimulation and enhancement. In the case of a modulator of a polypeptide, the modulator may be a direct modulator of the biological activity of the polypeptide, or it may be a modulator of the gene which encodes the polypeptide; in the latter case, the biological activity of the polypeptide is indirectly modulated by a modulator that affects the transcription or translation of the gene (and does not directly act on the polypeptide). Modulators can include AS fragments, siRNAs, ribozymes, polypeptides and small chemical molecules, inter alia.

The term “inhibitor” generally refers to a molecule which is capable of partially or fully inhibiting the biological activity of a gene or gene product. Similarly to a modulator, an inhibitor may be a direct inhibitor of the biological activity of a polypeptide, or it may be an inhibitor of the gene that encodes said polypeptide, as described. Examples of different types of inhibitors are, inter alia: nucleic acids such as AS fragments, siRNA, or vectors comprising them; polypeptides such as dominant negatives, antibodies, or, in some cases, enzymes; catalytic RNAs such as ribozymes and small chemical molecules.

The term “antibody” as used herein refers to IgG, IgM, IgD, IgA, and IgE antibody, inter alia. The definition includes polyclonal antibodies or monoclonal antibodies. This term refers to whole antibodies or fragments of antibodies, e.g. antibodies without the Fc portion, single chain antibodies, fragments consisting of essentially only the variable, antigen-binding domain of the antibody, etc. The term “antibody” may also refer to antibodies against nucleic acid sequences obtained by cDNA vaccination. The term also encompasses antibody fragments which retain the ability to selectively bind with their antigen or receptor and are exemplified as follows, inter alia:

(1) Fab, the fragment which contains a monovalent antigen-binding fragment of an antibody molecule obtainable by digestion of whole antibody with the enzyme papain to yield a light chain and a portion of the heavy chain;

(2) (Fab′)₂, the fragment of the antibody obtainable by treating the whole antibody with the enzyme pepsin without subsequent reduction; F(ab′₂) is a dimer of two Fab fragments held together by two disulfide bonds;

(3) Fv, defined as a genetically engineered fragment containing the variable region of the light chain and the variable region of the heavy chain expressed as two chains; and

(4) Single chain antibody (SCA), defined as a genetically engineered molecule containing the variable region of the light chain and the variable region of the heavy chain linked by a suitable polypeptide linker as a genetically fused single chain molecule.

By the term “epitope” as used in this invention is meant an antigenic determinant on an antigen to which the antibody binds. Epitopic determinants usually consist of chemically active surface groupings of molecules such as amino acids or sugar side chains and usually have specific three dimensional structural characteristics, as well as specific charge characteristics.

By the term “dominant negative peptide” (DNP) is meant a polypeptide encoded by a cDNA fragment that encodes for a part of a protein (see Herskowitz I.: Functional inactivation of genes by dominant negative mutations. Nature. 1987 Sep. 17-23;329(6136):219-22. Review; Roninson I B et al., Genetic suppressor elements: new tools for molecular oncology—thirteenth Cornelius P. Rhoads Memorial Award Lecture. Cancer Res. 1995 Sep. 15;55(18):4023). This peptide can have a different function from the protein from which it was derived. It can interact with the full protein and inhibit its activity or it can interact with other proteins and inhibit their activity in response to the full-length (parent) protein. Dominant negative means that the peptide is able to overcome the natural parent protein and inhibit its activity to give the cell a different characteristic like resistance or sensitization to killing, resistance or sensitization to insulin induced glucose uptake, or any cellular phenotype of interest. For therapeutic intervention the peptide itself may be delivered as the active ingredient of a pharmaceutical composition, or the cDNA can be delivered to the cell utilizing known methods.

The term “dominant positive peptide” (DPP) refers to peptides which are encoded by cDNA fragments that constitute a part of a protein, like DNPs, but their function is different; they can partially or fully mimic the activity associated with the corresponding parent protein (and thus possess the same activity).

“Dominant polypeptide fragment” (DPF) as used herein is the generic term used to encompass both dominant negative peptides and dominant positive peptides.

The isolation and preparation of DPFs can be accomplished by methods well known in the art, such as, inter alia: Achilles Heel Method (AHM) (see PCT publications WO 98/21366, WO 01/57189 and U.S. Pat. No. 6,057,111, co-assigned to applicants), Technical Knock-Out (TKO) (Deiss L P, Feinstein E, Berissi H, Cohen O, Kimchi A.: Identification of a novel serine/threonine kinase and a novel 15-kD protein as potential mediators of the gamma interferon-induced cell death. Genes Dev 1995 Jan. 1;9(1):15-30; Science 285 (1999): 299a), Genetic Suppressor Elements (GSE) (Roninson I B, Gudkov A V, Holzmayer T A, Kirschling D J, Kazarov A R, Zelnick C R, Mazo I A, Axenovich S, Thimmapaya R.: “Genetic suppressor elements: new tools for molecular oncology—thirteenth Cornelius P. Rhoads Memorial Award Lecture” Cancer Res 1995 Sep. 15;55(18):4023-8; Ossovskaya et al: Use of genetic suppressor elements to dissect distinct biological effects of separate p53 domains. Proc. Natl. Acad. Sci. USA 1996, 93: 10309-10314; Holzmayer et al: Isolation of dominant negative mutants and inhibitory antisense RNA sequences by expression selection of random DNA fragments. Nucleic Acid Research 1991, 20: 711-717; Gudkov et al: Isolation of genetic suppressor elements, inducing resistance to topoisomerase II-interactive cytotoxic drugs, from human topoisomerase II cDNA. Proc. Natl. Acad. Sci. USA 1993, 90: 3231-3135; Gudkov et al: Cloning mammalian genes by expression selection if genetic suppressor elements: Association of kinesin with drug resistance and cell immortalization. Proc. Natl. Acad. Sci. USA 1994, 91: 3744-3748; Roninson et al: Genetic suppressor elements: new tools for molecular oncology—thirteenth Cornelius P. Rhoads Memorial Award Lecture. Cancer Res 1995 Sep. 15;55(18):4023-8; and Dunn et al: Isolation of efficient anti-virals: genetic suppressor elements against HIV-1. Gene Therapy 1999, 6: 130-137.), and Selection Subtraction Approach (SSA) (see PCT patent publication No. WO 99/47643, co-assigned to applicants).

In one aspect of the present invention, the polypeptide is known to have an effect, preferably an adverse effect, in a disease state or condition.

By “disease state” is meant an alteration in the state of the body or any of its organs, or a disruption in the performance of its vital functions; an individual afflicted with such a disease state is unable to perform at least one of the functions he was able to perform before the onset of the disease state, and/or unable to function normally.

A further embodiment of the present invention provides for a process of obtaining a species which inhibits the activity of a polypeptide, comprising:

a) genetically engineering a cell so that the cell expresses an active polypeptide fragment of the polypeptide, so as to inhibit the activity of the endogenous polypeptide;

b) contacting the cell with the species; and

c) determining if the species competes with the active polypeptide fragment, and thus inhibits the activity of the endogenous polypeptide.

In one aspect of the present invention, the polypeptide is known to have an effect, such as an adverse effect, in a disease state or condition.

The species may be a chemical compound, a polypeptide, or a polynucleotide, all as defined above.

The term “genetically engineering” as used herein includes transducing, transfecting and infecting a cell with a polynucleotide or expression vector which causes a cell to express a heterologous polypeptide, or any other manipulation which can cause a cell to express a heterologous polypeptide.

The term “expression vector” as used herein refers to vectors that have the ability to incorporate and express heterologous DNA fragments in a foreign cell. Many prokaryotic and eukaryotic expression vectors are known, and/or commercially available. Selection of appropriate expression vectors is within the knowledge of those having skill in the art.

An additional aspect of the present invention provides for the use of a species identified according to the methods described above, for the preparation of a medicament.

Another aspect of the present invention concerns a method of preparing a pharmaceutical composition, comprising identifying a species which inhibits the activity of a polypeptide according to any one of the methods described above, and admixing the identified species or a chemical analog or homolog thereof with a pharmaceutically acceptable carrier.

By “chemical analog” as used herein is meant a molecule derived from the originally identified inhibiting species, that retains the inhibitory activity, more particularly that retains the specific inhibitory activity observed in the parent molecule; chemical analogs may also share structural properties with the parent inhibiting species.

A further embodiment of the present invention provides a method of treating a subject in need thereof comprising administering to the subject the pharmaceutical composition prepared according to the above method, in a dosage sufficient to treat the subject; the subject may be suffering form an apoptosis related disease such as, inter alia: cancer, a neurological disorder/neurodegenerative disease, for example: stroke, Parkinson's disease, epilepsy, depression, dementia, ALS (Amylotrophic lateral sclerosis), Alzheimer's disease, Huntington's disease. Additional diseases which the pharmaceutical compositions of the present invention can be employed to treat include cardiovascular diseases, ischemic conditions and ischemia-reperfusion injuries, fibrotic conditions such as kidney or liver fibrosis and metabolic diseases, inter alia.

By “apoptosis related disease” is meant, in general, any disease that involves abnormal apoptosis of cells. Abnormal means typically at least 10% preferably 20%, 30%, 40% or 50% lower or higher than normal. Examples of apoptosis-related diseases are, inter alia, cancer and neurodegenerative diseases.

The term “apoptosis” is particularly defined as execution of built-in cell death program resulting in chromatin fragmentation into membrane-bound particles, changes in cell cytoskeleton and membrane structure and subsequent phagocytosis of apoptotic cell by other cells. However, as used herein, it should be understood that this term should be construed more broadly as encompassing cell death, whether or not that cell death is strictly by means of the apoptotic process described above.

An additional embodiment of the present invention concerns a method of screening a microarray comprising:

a) contacting a microarray with a library of active polypeptide fragments; and

b) examining the microarray for a positively reacting species.

This method may include the additional steps of:

c) isolating the species which reacted positively;

d) identifying the active polypeptide fragment which caused said species to react;

e) examining the effect of the species on the parent polypeptide

The microarray may be imprinted with chemical compounds, polypeptides or polynucleotides; in addition, the active polypeptide fragments of the library may each be tagged, including uniquely tagged, according to methods well known in the art (see for example co-assigned PCT publication WO 99/47643). Methods of tagging polypeptides, including active polypeptide fragments, are known in the art. For further details see Example 8.

In a related embodiment of the present invention, a kit for performing the aforesaid method is provided. This kit comprises:

a) a microarray; and

b) a library of active polypeptide fragments.

The microarray may be imprinted according to the specifications of the method to be carried out (with chemical compounds, polypeptides or polynucleotides); in addition, the active polypeptide fragments of the library may be tagged or uniquely tagged.

An additional embodiment of the present invention concerns a process of obtaining a species that modulates a phenotype comprising:

(a) contacting a microarray comprising an array of species with a plurality of active polypeptide fragments;

(b) examining the microarray for a positively reacting species

(c) obtaining one or more species which reacted positively; and

(d) examining the effect of the species of step (c) on a cell exhibiting a phenotype.

This process may additionally comprise:

(e) identifying the active polypeptide fragment which caused the species to react;

(f) obtaining a parent polypeptide; and

(g) examining the effect of the species on the parent polypeptide.

In an alternative embodiment, the above process may additionally comprise:

(e) identifying the active polypeptide fragment which caused the species to react;

(f) obtaining a parent polypeptide; and

(g) examining the effect of the parent polypeptide on the phenotype.

It will be understood that these processes can in addition be performed (according to step (a)) with one or more active polypeptide fragment as a probe (and not necessarily a plurality).

In related embodiments, one or more of the plurality of active polypeptide fragments of step (a) are associated with the phenotype exhibited by the cell of step (d). In addition, the active polypeptide fragments of step (a) may be tagged or uniquely tagged.

By “associated with a phenotype” is meant known or suspected to be involved in the phenotype by any particular mechanism.

The phenotype in question may be present in a disease, such as the diseases listed herein, or may even be a model phenotype for a particular disease. For example, inter alia, a tumor cell as a model for a cancer, an oxygen deprived cell as a model for a degenerative disease, a cell lacking a particular gene (e.g., a knockout) as a model for a disease in which said gene is lacking (or the polypeptide encoded thereby mutated or inactive), etc.

The species imprinted on the microarray used to perform the above processes may be chemical compounds, polypeptides, polynucleotides or other molecules encompassed by the term species, as defined above.

Further to the above processes, an additional aspect of the invention provides a kit for performing said processes which comprises:

(a) a microarray; and

(b) a plurality of active polypeptide fragments.

The plurality of active polypeptide fragments may be associated with a phenotype and/or a disease. For example, the invention provides a kit for obtaining one or more species that modulate cancer comprising a microarray and a plurality of active polypeptide fragments known or suspected to be involved in cancer or a cancerous phenotype; the active polypeptide fragments may be randomly prepared from cancerous cells, or, alternatively, may be derived from one or more polypeptides known to have a role in cancer or the cancerous transformation of a cell. By the same token, active polypeptide fragment libraries can be prepared for any known disease, and combined with a microarray imprinted with any kind of species to from a kit which may be used for performing the above processes and thereby obtaining species which modulate said diseases and/or phenotype; these kits are considered part of the present invention.

By “library” in the context of an active polypeptide fragment library, is meant preferably at least 5 active polypeptide fragments which differ from each other. A library may be prepared from random APFs, APFs derived from the same polypeptide, APFs derived from homologous polypeptides or polypeptides from the same family, or APFs known or suspected to be involved in a specific phenotype or disease. Additionally, said library may comprise different types of APFs, as defined above.

By “parent protein/parent polypeptide” as used herein is meant the endogenous protein/polypeptide that corresponds to the active polypeptide fragment or an allelic variant or homolog thereof.

By “homolog/homology”, as related to polynucleotides and polypeptides and used herein, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these polynucleotides and polypeptides can be used in the same fashion as the herein or aforementioned polynucleotides and polypeptides.

Alternatively or additionally, “homology”, with respect to sequences, can refer to the number of positions with identical nucleotides or amino acid residues, divided by the number of nucleotides or amino acid residues in the shorter of the two sequences, wherein alignment of the two sequences can be determined in accordance with the Wilbur and Lipman algorithm ((1983) Proc. Natl. Acad. Sci. USA 80:726), for instance, using a window size of 20 nucleotides, a word length of 4 nucleotides, and a gap penalty of 4, and computer-assisted analysis and interpretation of the sequence data, including alignment can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil. (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J. Molec. Evol. 25:351-360; Higgins et al., (1989) CABIOS 5:151-153; and Thompson et al., (1994) Nucl. Acids Res. 22:4673-4680.

An additional embodiment of the present invention relates to the ability to identify chemicals that bind to a specific site within a polypeptide. Chemical molecules that bind to an active site of a polypeptide may be located by comparing chemical binding profiles between a normal polypeptide and a polypeptide obtained by site-directed (or random) mutagenesis in an active site of the aforementioned polypeptide, by “subtracting” the total chemical molecule binding profile of the mutated polypeptide from that of the normal one. Individual inhibitors specific for an active site or that inhibit a biological activity of a polypeptide have been previously researched by comparing the binding of a specific molecule to both the normal and active-site-mutated counterpart of a polypeptide (see for example: PNAS USA 90: 5133-5137, 1993; The Journal of Biological Chemistry, 277 (29): 26422-26428, 2002; The Journal of Biological Chemistry 270 (10): 5057-5064, 1995; The Journal of Biological Chemistry 268 (33): 24572-24579, 1993; The Journal of Biological Chemistry 274 (28): 15706-15711, 1999; Journal of Virology 72(3): 2456-2462, 1998; Cancer Research 57(8): 1516-1522, 1997.). Mutagenesis can be carried out as known in the art and essentially as described in “Molecular Biology: Current Innovations and Future Trends”, Horizon Scientific Press, A. M. Griffin and H. G. Griffin (Eds.), Norwich, UK (October 1995). Another advantage of this embodiment, in addition to locating specific inhibitors, is that inhibitors can be found for “non-druggable” polypeptides that have no known (or currently detectable) enzymatic activity, so long as binding activity of some type can be measured. This is because the inhibitor identification is based on binding and not biological activity; this feature can facilitate high throughput screening assays with such polypeptides. This embodiment therefore concerns a process of obtaining a small-molecule polypeptide inhibitor comprising:

a) screening a plurality of small molecules with a polypeptide probe;

b) screening the plurality of small molecules of step a) with a second polypeptide probe comprising the polypeptide probe of step a) further comprising one or more amino acid mutation;

c) comparing the small molecule binding profile of the probe of step b) to that of the probe of step a); and

d) identifying one ore more small molecules from the plurality of small molecules that bind to the probe of step a) but not to the probe of step b).

The plurality of small molecules may be imprinted on a microarray or on one or more bead of any type. The mutation of step b) may be in a region of the polypeptide known to be involved in a biological activity such as, inter alia, a biological activity selected from the biological activity group consisting of: enzymatic activity, protein-protein interaction, protein-DNA interaction, protein-carbohydrate interaction, protein-chemical interaction, protein modification, localization signal, ATP/GTP carrying site, and ion binding. Possible biological activity sites that may be examined for chemical molecule binding include, inter alia, protein-sugar interaction sites, glycosylation (or any other protein modification) sites; NLS and other localization signal sites; known protein-chemical interaction sites (wherein the chemical is not useful for treatment); ATP/GTP carrying sites (wherein there is no ATPase or GTPase activity); zinc or other metal binding sites. The mutation in the polypeptide probe of step b) may comprise a series of mutations, which can be in any amino acid comprising an active site—to any amino acid, whether functionally similar or dissimilar (distant in terms of amino acid type or family). Further, the mutation may be site directed or randomly generated. In the case of a randomly generated mutation, it is possible to use the process in order to identify area of the polypeptide which are crucial for the binding of a particular chemical compound, and the relation between the site, the compound and the activity of the polypeptide (e.g., loss of activity as a result of binding to the small molecule is measured). Any form of the polypeptide, whether a full polypeptide, a fragment of the full polypeptide or a truncated polypeptide, whether naturally purified or synthetically produced may be used, provided that it maintains the original conformation of an active site of the native polypeptide.

By the term “biologically active” or “biological activity” as used herein, is meant the ability of a molecule such as a polypeptide or polynucleotide to execute, influence or modulate a biological process.

As described, the above embodiment of the present invention provides for the use of a mutated polypeptide in which an amino-acid in the active domain is changed, thus rendering the polypeptide inactive (with respect to the activity performed through said active domain). The binding of the normal polypeptide to chemical molecules will be compared to the binding of the mutated polypeptide. The profile of chemical molecules that bind to the normal and mutated polypeptides will allow the selection of two groups of chemical molecules:

1. Those that preferentially bind to the normal polypeptide; and

2. Those that preferentially bind to the mutated polypeptide.

These groups of chemical molecules may subsequently be enriched for those that inhibit the polypeptide. An inhibiting chemical compound will likely bind to the normal polypeptide but not to the mutated polypeptide. Several mutations in various positions in one or more active domains can be compared to obtain an accurate profile of chemical binding. The greater the change in the chemical molecules that bind the mutated polypeptide in comparison to the normal, the higher the probability that the mutation caused a conformational change in the polypeptide; preferred chemical molecules are those for which a differential is achieved (no binding in the mutated polypeptide vs. binding in the normal polypeptide), wherein the rest of the binding profile is similar amongst both polypeptides (meaning that the change the mutation caused was local and not global).

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including U.S. patents, are referenced by author and year and patents by number. The disclosures of these publications and patents and patent applications in their entireties are hereby incorporated by reference into this application in order to more fully describe the state of the art to which this invention pertains.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol. 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

### Example 1

### Preparation of Active Polypeptide Fragments

The active polypeptide fragments used for screening in the present invention can be prepared by the following methods:

A. Preparation of Active Polypeptide Fragments Comprising an Active Site of a Particular Known Gene/Polypeptide

In order to prepare APFs derived from a particular gene, the areas of the gene that are of interest can be cloned in an expression vector and the vector subsequently delivered to a host cell for expression. A series of APFs may be prepared, spanning different regions of a gene or even different portions of a particular active site. In addition, the desired APFs can be synthesized directly using a commercially available machine, as detailed in Example 3. Further, APFs can be prepared by random fragmentation of the parent polypeptide through methods known in the art (e.g., mechanical fragmentation, chemical digestion, sonication, etc.). The polypeptide fragments can be chemically or synthetically modified in order to increase their stability or activity. In addition, they may be modified so as to preserve the conformation of the active site as it is present in the naturally occurring polypeptide.

B. Preparation of Dominant Negative Peptides

1. Isolation of Dominant Negative Peptides Based on Functional Selection

The isolation of DNPs (or DPPs) based on functional selection can be accomplished by several methods well known in the art; below follows a detailed example of the GIE (“Genetic Inhibitory Element”) approach; this example also combines steps of the AHM technique. Additionally, the methods of SSH, SSA, TKO and AHM can be employed; these methods are referenced above and are incorporated herein by reference.

GIEs are short, biologically active gene fragments, which can be generated by random fragmentation of target genes. The GIE, which is a DNA fragment, matches a part of an endogenous gene. In a cell, GIEs may be transcribed into mRNA. When compared to the mRNA of the endogenous gene, GIE-derived mRNA can be either in antisense orientation or in the sense orientation. When in an antisense orientation the GIE-derived mRNA can inhibit the expression of the endogenous mRNA. When in the sense orientation, the GIE-derived mRNA can encode a short polypeptide that possesses the same activity as the matching endogenous protein.

a. Preparing a Library of GIEs

In order to prepare a cell library of GIEs, total cDNA is prepared from a cell of interest. cDNA fragments of a length range of approximately 200 to 600 bp (but not limited to this size range) derived from the total cDNA are introduced into an expression vector (for cloning and other routine DNA manipulations see Sambrook et al., Molecular Cloning: A Laboratory Manual, Cold Springs Harbor Laboratory, New York (1989, 1992), and Ausubel et al., Current Protocols in Molecular Biology, John Wiley and Sons, Baltimore, Md. (1989)). Such a library is a collection of many (usually 100,000 to 1,000,000) different GIE containing expression vectors. The expression vector also includes a gene encoding a protein that confers resistance to “drug A”. The expression vector library, harboring the GIEs, is then introduced into cultured cells. The entire cell culture is then exposed to appropriate selection (using “drug A”), and only cells that contain an expression vector harboring a GIE and the drug resistance gene, remain alive and keep multiplying. This resulting cell culture is termed a “cell library”.

In each cell of the cell library a few copies of the vector exist. In the case of episomal vectors, it was found that within one cell, all copies of the vector have the same cDNA fragment (in most cases). The cDNA fragment in the vector, the GIE, is expressed, namely, transcribed into mRNA. The cDNA fragment, the GIE, in the vector matches a part of an endogenous gene. When compared to the mRNA of the endogenous gene, GIE-derived mRNA can be either in antisense orientation or in the sense orientation. When in an antisense orientation, the GIE-derived mRNA can inhibit the expression of the endogenous mRNA. When in the sense orientation, the GIE-derived mRNA can encode a short polypeptide that can be a competitor of the matching endogenous protein. Such a short polypeptide is termed dominant negative peptide. Both posibilities lead to inhibition of the expression of the specific endogenous gene. Importantly, if in a given cell the expressed GIE inhibited a gene essential for general cell viability, such a cell will die or will stop multiplying and will quickly decrease in abundance. For example, if the mRNA encoding DNA polymerase II is inhibited, then the cell will not be able to multiply. In the cell library every cell contains a vector expressing a GIE. Thus, in each cell the function of a specific, and different; gene can be inhibited either by the inhibition of the mRNA by antisense mRNA or by inhibition of the protein by a dominant negative peptide.

b. Applying Functional Selection to the Library of GIEs

Following the establishment of the cell library, an aliquot of the cells is exposed to a selection process that can be followed experimentally. That is, the cells are exposed to conditions requiring/activating a phenotype of interest. Such a phenotype includes, but is not limited to, activation of cell-death, causing growth arrest, activation of contact inhibition, appearance or disappearance of specific markers (proteins or other distinctive chemical groups), activation or repression of specific promoters. The conditions requiring/activating the phenotype include, but are not limited to, treating cells with specific proteins, chemicals, antibodies, antisense oligonucleotides, exposing the cells to specific, special, growth conditions such as limiting oxygen levels, limiting amounts of nutrients in growth medium, and growing the cells to specific cell density. A reserved aliquot of the cell library is not exposed to the selection. Thus, if the expression of a key gene is inhibited, the phenotype does not show for that cell or shows only for that cell, depending on the function of the endogenous gene. The selection process is followed by identification of the GIE that was present in the expression vector that was found in the cell. The identity of this GIE is indicative of the identity of the inhibited gene, thus identifying it as a key gene required for the change in phenotype.

c. Identifying Resultant GIEs that Encode Dominant Negative Peptides

Based on the above selection procedures, GIEs that inhibit genes which are required for selected changes in a phenotype of the cell are identified and isolated (for specific methods of identification and isolation of functional cDNAs, see the above references). The GIE of interest is then validated to encode a dominant negative peptide. This validation is based on bioinformatic analysis indicating that the GIE of interest has an open reading frame and can be translated into a polypeptide, and that it falls within the open reading frame of the endogenous gene. This bioinformatic analysis can be validated by mutating the selected GIE, so that it can no longer be translated into a polypeptide, and examining whether it retains its inhibitory activity on the endogenous polypeptide (if the mutation causes the GIE to lose its inhibitory activity, it is likely to be a dominant negative peptide).

d. Generating Dominant Negative Peptides from the Selected GIEs

After the GIEs of interest are found to affect a certain cellular phenotype of interest, and are shown to encode dominant negative peptides, these dominant negative peptides can be prepared by methods well known in the art. For preparation of polypeptides see Example 2.

2. Generation of Dominant Negative Peptides for a Gene of Interest

The same process as described above can be carried out on a gene or several genes of interest instead of a library of total cellular cDNA. If a gene or genes of interest are already known to have a certain activity related to a phenotype of interest, the process may be slightly different. In this case, cDNA is prepared from the gene or genes of interest, fragmented, and fragments of a desired size are inserted into expression vectors. The cDNAs must then be screened to identify those that encode polypeptides (as described in step c. above) and inhibit the activity of the parent polypeptide (in a cell based functional selection system, as described above; or in a cell free system using a known activity assay of the parent polypeptide, such as an enzymatic activity assay), and are thus dominant negative peptides for the gene or genes of interest.

For references on how to prepare dominant negative peptides/GIEs see: Gudkov et al. (1994) Cloning mammalian genes by expression selection of genetic suppressor elements: association of kinesin with drug resistance and cell immortalization. Proc. Natl. Acad. Sci. U.S.A., 91:3744-3748.; and Holzmayer et al. (1992) Isolation of dominant negative mutants and inhibitory antisense RNA sequences by expression selection of random DNA fragments. Nucleic Acids Res., 20:711-717.

### Example 2

### Preparation of Polypeptides

Polypeptides may be produced via several methods, for example:

1) Synthetically;

Synthetic polypeptides can be made using a commercially available machine, using the known sequence of the desired polypeptide.

2) Recombinant Methods:

A preferred method of making polypeptides is to clone a polynucleotide comprising the cDNA of the gene of the desired polypeptide into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965;23:1165-74 Appl Microbiol. 1967 July;15(4):851-6; Can J Biochem. 1968 May;46(5):441-4; Biochemistry. 1968 July;7(7):2574-80; Arch Biochem Biophys. 1968 Sep. 10;126(3):746-72; Biochem Biophys Res Commun. 1970 Feb. 20;38(4):825-30).). The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).

3) Purification from natural sources:

Desired polypeptides can be purified from natural sources (such as tissues) using many methods known to one of ordinary skill in the art, such as for example: immunoprecipitation, or matrix-bound affinity chromatography with any molecule known to bind the desired polypeptide.

Protein purification is practiced as is known in the art as described in, for example, Marshak et al., “Strategies for Protein Purification and Characterization. A laboratory course manual.” CSHL Press (1996).

Active polypeptide fragments can be prepared according to the above principles detailed in this example, in particular in sections 1 and 2; for further detail see also Example 1.

### Example 3

### Preparation of Polynucleotides

The polynucleotides of the subject invention can be constructed by using a commercially available DNA synthesizing machine; overlapping pairs of chemically synthesized fragments of the desired gene can be ligated using methods well known in the art (e.g., see U.S. Pat. No. 6,121,426).

Another means of isolating a polynucleotide is to obtain a natural or artificially designed DNA fragment based on that sequence. This DNA fragment is labeled by means of suitable labeling systems which are well known to those of skill in the art; see, e.g., Davis et al. (1986). The fragment is then used as a probe to screen a lambda phage cDNA library or a plasmid cDNA library using methods well known in the art; see, generally, Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Md. (1989),

Colonies can be identified which contain clones related to the cDNA probe and these clones can be purified by known methods. The ends of the newly purified clones are then sequenced to identify full-length sequences. Complete sequencing of full-length clones is performed by enzymatic digestion or primer walking. A similar screening and clone selection approach can be applied to clones from a genomic DNA library.

### Example 4

### Preparation of Microarrays

The specific details of the different microarray preparation processes (different methods and, in addition, binding of nucleic acids, proteins, and compounds to the microarrays) can be found in: Schena et al., Parallel human genome analysis: Microarray-based expression monitoring of 1000 genes. Proc. Natl. Sci. USA (1996) 93, 10614-10619; U.S. Pat. Nos. 6,291,170 and 5,807,522 (see above); U.S. Pat. No. 6,037,186 (Stimpson, inventor) “Parallel production of high density arrays”; PCT publications WO 99/13313 (Genovations Inc [US], applicant) “Method of making high density arrays”; WO 02/05945 (Max-Delbruck-center for molecular medicine [Germany], applicant) “Method for producing microarray chips with nucleic acids, proteins or other test substrates”.

### Example 5

### Screening of Microarrays

Nucleic acid microarray analysis is performed essentially as described in the references incorporated herein. See, for example, U.S. Pat. Nos. 6,291,170; 5,807,522; 6,110,426; 5,716,785; 5,891,636 and 4,981,783.

Polypeptide microarray analysis is performed essentially as described in the references incorporated herein. See for example: Ekins et al. J Pharm Biomed Anal 1989. 7: 155; Ekins and Chu, Clin Chem 1991. 37: 1955; Ekins and Chu, Trends in Biotechnology, 1999, 17, 217-218; MacBeath and Schreiber, Science 2000; 289(5485): p.1760-1763.

Chemical microarray analysis is performed essentially as described in the references incorporated herein. See for example: Gregory et al.: Journal of the American Chemical Society; 2001; 123(2); 361-362; Hergenrother et al., Journal of the American Chemical Society; 2000; 122(32); 7849-7850; MacBeath et al., Journal of the American Chemical Society; 1999;121(34); 7967-7968; Kuruvilla et al (Nature 416 (2002), 653-657.

### Example 6

### Validation

Validation of the species identified according to the present invention can be done by binding or activity assays, according to biochemical methods known in the art.

Specifically, the first step of the validation process is to verify that the species identified actually binds to the active polypeptide fragment used in the screen. (If a plurality of peptides were used, the specific peptide which binds to the species must first be identified).

This can be checked with the help of several different assays well known in the art, and the assay selection is contingent on the identity of the species identified. For example: if the active polypeptide fragment has a known activity, the effects of the isolated species on this activity can be examined according to an appropriate enzymatic assay. If, on the other hand, protein-protein or protein-nucleic acid interactions are to be tested, there are appropriate methods known in the art.

One such method, which tests binding between interacting molecules, is a gel-shift assay, which is based on the fact that bound molecules will travel more slowly then unbound molecules of the same type during electrophoresis through a gel matrix. If the binding reaction causes the molecule to be retarded in the gel as compared to the unbound molecule, then it was successful.

The second step of the validation process entails examining the inhibitory (or agonistic) effect of the identified species on the parent protein that corresponds to the active polypeptide fragment; again, this validation can be performed according to enzymatic assays known in the art. In addition, if the parent protein causes or diminishes a certain cellular phenotype, cells expressing the parent protein (which may be genetically engineered to do so) can be treated with the species, in order to examine whether the species affects the exhibited cellular phenotype.

In addition, in case of a polynucleotide probe, the effect of an identified small chemical molecule on the transcription or protein expression of the endogenous gene (i.e., the parent protein) can be examined in-vivo in an appropriate cell culture system.

In the case of validation of the effect of an identified species on a phenotype, the species may be re-tested in the cell system used to perform the process and, in addition, in several other cell types which are relevant for a particular phenotype being tested. In addition and in the case of a cell which lacks a particular polypeptide, the species may be tested as a control on the same cell type which has normal expression of said polypeptide. Further, the direct interaction between the identified polypeptide and said species may also be examined according to methods known in the art, as described herein.

### Example 7

### Identification and Isolation of Genes Based on cDNA Fragments which Encode the APFs

The genes from which the original active polypeptide fragments were derived can be identified (if necessary) by bioinformatic analysis using the available software and databases. Should this approach fail, the genes may be identified by molecular biology methods well known in the art, such as screening of different types of genomic and cDNA libraries using the original cDNA encoding the APF as a probe, or 5′ race reactions using the cDNA sequence as a template.

For specifics of these and additional methods see, for example: Sambrook et al., Molecular cloning: A laboratory manual, Cold Springs Harbor Laboratory, New-York (1989, 1992); Ausubel et al., Current Protocols in Molecular Biology, John Wiley and Sons, Baltimore, Md. (1989).

### Example 8

### Screening a Small Chemical Molecule Microarray with a Polypeptide Probe

As described herein, one of the methods of the present invention provides means for identifying chemical molecules that inhibit the biological activity of a polypeptide. Said method, as described above, is carried out by comparing small molecule binding profiles of a normal (wild-type) and corresponding mutated polypeptide probe.

Small molecule microarray analysis with one or more polypeptide probes is performed essentially as described below and in the references incorporated herein.

Probes

The conditions of the binding reaction between the polypeptide probe and the microarray should be tailored to the polypeptide being used. In addition to the standard methods known in the art for screening a small molecule microarray with a polypeptide probe (some of which are referenced above), It is important to recognize that most polypeptides have some capacity to adsorb onto almost any surface, due to localized hydrophobic, hydrophilic, and ionic domains on the surface of the polypeptide. This ability to adsorb non-specifically can be minimized by using sufficient concentrations (e.g., 1-10 mg/ml) of an inert, non-detectable protein such as bovine serum albumin (BSA) or casein in wash buffers and during subsequent incubation with dye-labeled protein probes.

Detection Systems

In order to effectively screen small molecule microarrays, a system to specifically detect binding of the probe to a small molecule is necessary. One detection system employed with the present invention is the measurement of fluorescence emanating from dye-conjugated probes, in several possible variations. For each probe being used in the screening methods of the present invention, it is necessary to conjugate the dye in a manner that does not interfere with binding. Several examples are: 1. performing the dye labeling in the presence of an excess of the small molecule, provided that it does not have amino groups. 2. biotinylating the probe (kits for performing such modification are available, for example, from Roche or Calbiochem) and following with fluorescent-labelled streptavidin. 3. performing the dye labeling on an antibody complementary to the polypeptide probe, and measuring fluorescence of the immunological complex.

The dyes Cy3, Cy5, and Alexa, inter alia, are conjugated to the probe as known in the art; reagents, protocols, and complete kits needed to conjugate these dyes to polypeptides, including antibodies, are available from several manufacturers including BioRad, Molecular Probes, and Amersham Pharmacia Biotech.

An additional option for fluorescent detection is constructing fusion proteins of a desired polypeptide probe with green fluorescent protein (GFP).

The scanning of microarrays for fluorescence emission may be performed with an Axon Instruments Model 4000 fluorescent scanner; the data is processed using GenePix software.

Additional detection systems include, inter alia: radioactively tagging the probe with an appropriate isotope according to methods known in the art; alkaline phosphatase assay, horse-radish peroxidase assay, and immuno-detection with specific antibodies.

Possible Re-Cycling

In order to re-cycle the small molecule microarray for re-probing, the previous probe must be stripped from the microarray. This can be accomplished according to methods known in the art, depending on the type of probe that needs to be stripped.

Validation

Validation of the binding between the chemical molecule on the microarray and the polypeptide probe can be accomplished by methods known in the art, some of which are described in Example 6 above.

The small molecule that bound to the polypeptide probe can be identified according to its location on the microarray, as every well contains only one type of molecule; following the identification, the small molecule can be prepared (or extracted from the original library) and a validation binding assay can be conducted in vitro between the polypeptide probe and the small molecule, under the appropriate conditions and with the same detection system as in the case of the entire microarray, or with other detection systems known in the art.

In addition, the effect of the identified small molecule on the biological activity of the polypeptide probe can be examined. A known activity of the polypeptide can be measured before and after/with or without binding to the small molecule. Detectable biological activities are described herein and include enzymatic activities, binding, localization, modification and more.

### Example 9

### Delivery of Chemical Compounds

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred. In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantities to be administered vary for the patient being treated and vary from about 100 ng/kg of body weight to 100 mg/kg of body weight per day and preferably are from 10 μg/kg to 10 mg/kg per day.

In addition, the compound or pharmaceutical composition of the present invention can be formulated and administered to a patient as a pro-drug.

1. A process of obtaining a species which inhibits the activity of a polypeptide, comprising:
a. contacting an active polypeptide fragment of the polypeptide with the species;
b. determining if the species interacts with the active polypeptide fragment; and
c. determining if the species inhibits the activity of the polypeptide.

2. The process of claim 1 wherein the species is a chemical compound.

3. The process of claim 1 wherein the species is a polypeptide.

4. The process of claim 1 wherein the species is a polynucleotide.

5. The process of claim 1 wherein the species is imprinted on a microarray.

6. The process of claim 1 wherein the polypeptide is known to have an effect in a disease state or condition.

7. The process of claim 1 wherein the species is a chemical compound imprinted on a microarray and the active polypeptide fragment is a dominant polypeptide fragment.

8. The process of claim 1 wherein the species is a chemical compound imprinted on a microarray and the active polypeptide fragment is a polypeptide fragment comprising an active site of one or more polypeptide of interest.

9. A process of obtaining a species that modulates a phenotype comprising:
(a) contacting a microarray comprising an array of species with a plurality of active polypeptide fragments;
(b) examining the microarray for a positively reacting species;
(c) obtaining one or more species which react positively; and
(d) examining the effect of the species of step (c) on a cell exhibiting the phenotype.

10. The process of claim 9 further comprising:
(e) identifying the active polypeptide fragment which caused the species to react;
(f) obtaining a parent polypeptide; and
(g) examining the effect of the species on the parent polypeptide.

11. The process of claim 9 further comprising:
(e) identifying the active polypeptide fragment which caused the species to react;
(f) obtaining a parent polypeptide; and
(g) examining the effect of the parent polypeptide on the phenotype.

12. The process of claim 9 wherein one or more of the active polypeptide fragments of step (a) are associated with the phenotype exhibited by the cell of step (d).

13. The process of claim 9 wherein the active polypeptide fragments of step (a) are tagged.

14. The process of claim 9 wherein the species imprinted on the microarray are chemical compounds.

15. The process of claim 9 wherein the species imprinted on the microarray are polypeptides.

16. The process of claim 9 wherein the species imprinted on the microarray are polynucleotides.

17. A kit for performing the process of claim 9 which comprises:
a. a microarray; and
b. a plurality of active polypeptide fragments.

18. The kit of claim 17 wherein the plurality of active polypeptide fragments are associated with a phenotype.

19. A process of obtaining a small molecule polypeptide inhibitor comprising:
a) screening a plurality of small molecules with a polypeptide probe;
b) screening the plurality of small molecules of step a) with a second polypeptide probe comprising the polypeptide probe of step a) further comprising one or more amino acid mutation;
c) comparing the small molecule binding profile of the probe of step b) to that of the probe of step a); and
d) identifying one or more small molecules from the plurality of small molecules that bind to the probe of step a) but not to the probe of step b).

20. The process of claim 19 wherein the plurality of small molecules is imprinted on a microarray.

21. The process of claim 19 wherein the mutation of step b) is randomly generated.

22. The process of claim 19 wherein the mutation of step b) is in a region of the polypeptide known to be involved in a biological activity.

23. The process of claim 22 wherein the biological activity is selected from the biological activity group consisting of: enzymatic activity, protein-protein interaction, protein-DNA interaction, protein-chemical interaction, protein-carbohydrate interaction, protein modification, localization signal, ATP/GTP carrying site, and ion binding.

24. The process of claim 22 wherein the mutation of step b) is in a region of the polypeptide known to be involved in an enzymatic activity.
